(19) United States
(12) Patent Application Publication
(10) Pub. No.: US 2012/0231533 A1
(43) Pub. Date: Sep. 13, 2012

(54) DEVICE AND METHOD FOR THE STUDY OF CELL AND TISSUE FUNCTION

(76) Inventors: Mark R. Holl, Tempe, AZ (US); Deirdre R. Meldrum, Phoenix, AZ (US); A. Cody Youngbull, Tempe, AZ (US); Haixin Zhu, Chandler, AZ (US); Jeff Houkal, Tempe, AZ (US); Yanqing Tian, Chandler, AZ (US); Shashanka Ashili, Phoenix, AZ (US); Laimonas Kelbauskas, Gilbert, AZ (US); Roger Johnson, Phoenix, AZ (US); Shih-hui Chao, Phoenix, AZ (US); Peter Wiktor, Phoenix, AZ (US); Alex Jen, Kenmore, WA (US); Lloyd Burgess, Seattle, WA (US); Sarah McQuaid, Seattle, WA (US); Ai Brunner, Phoenix, AZ (US); Peter Kahn, Phoenix, AZ (US)

(21) Appl. No.: 13/126,700

(22) PCT Filed: Oct. 28, 2009
(86) PCT No.: PCT/US2009/062380

§ 371 Date: Jul. 12, 2011

Related U.S. Application Data
(60) Provisional application No. 61/108,998, filed on Oct. 28, 2008.

(51) Int. Cl. C12M 1/34

(52) U.S. Cl. 435/287.9; 435/288.7

(57) ABSTRACT

A chamber device for analyzing living cell(s). The chamber device includes a base and a lid that when reversibly pressed closed create a chamber. The base is configured with an optically transparent well to contain at least one cell. The lid has a breadth greater than the base and is configured to contain at least one sensor. The lid is further configured with a lip that when pressed between the lid and the base creates an impermeable seal. The base and the lid are configured so that, when closed and in use, the sensor remains spatially apart from the at least one cell.

### FEDERAL FUNDING LEGEND

This invention was made with government support under Grant No. 5P50 HG002360-08 awarded by the NIH/NHGRI. The government has certain rights in the invention.

### FIELD OF THE INVENTION

The present invention relates to automated laboratory equipment. More specifically, the present invention provides devices and methods that allow the observation and measurement of parameters of interest both inside and outside the confines of living cells and tissue, allowing the analysis of microenvironmental physiological response.

### BACKGROUND OF THE INVENTION

Laboratory automation is a classic instance of high throughput automation. It is a rapidly developing technology which poses several difficult challenges such as high throughput, efficient information management, multi-disciplinary automation tasks, to name a few. Cellular analysis has emerged as the predominant avenue for laboratory automation. More specifically, research on single cells includes high throughput procedures such as cell selection, real-time data acquisition for stimulus/response experiments, and end point analyses such as PCR. These analyses require high precision in operation and measurement, and also generate large volumes of data. In order to achieve these objectives, a novel method for constructing a microenvironment or a plurality of microenvironments has been conceived and demonstrated.

### SUMMARY OF THE INVENTION

The devices and methods of the present invention provide for the measurement of intracellular and extracellular physiological response of living cells to external stimuli using optical or electronic sensor transduction means. An embodiment of the present invention provides for an automated system that places one or more cells or a tissue sample in an chamber that can alternately be opened or closed. The chamber can be perfused when open with any media or stimulus of choice. When closed, the chamber is sealed and the depletion of, or accumulation of, moieties of interest can be observed using sensors or sensor chemistries within the chamber. As such, the chamber enables the measurement of metabolic rates of production and consumption. For example, when the chamber materials are impermeable to oxygen and other gases, gas consumption and production rates can be measured. In other embodiments, the chamber lid material may be permeable to oxygen and impermeable to other moieties of interest (e.g., extracellular proteins) and therefore can be used to measure the buildup of these with appropriate sensor selections.

The present invention enables the performance of analysis techniques using novel device geometries, novel integration of manufacturing process technologies, novel use of patterned material functionalizations and coatings, novel utilization of sensor deposition techniques, novel methods of device cassette manufacturing for automation, novel means of cell placement, and novel methods of measurement scanning Novelty is derived for each individual innovative improvement. This novelty is significantly amplified by the plurality of permutations offered within the invention—as well as by the unique enablement of a previously not attainable dynamic range in the detection of moieties of interest.

### BRIEF DESCRIPTION OF THE DRAWINGS

FIGS. 1A and 1B depict an alternative assay format that creates a chamber in which individual cells may be sealed from external exposure using a base, lid, and piston system.

FIG. 2 depicts an aspect in which the base is treated with a cell-promoting substance, and areas external to the chamber are treated with non-fouling substances.

FIG. 3 shows non-fouling coatings that may be applied to the lid of the device, and point out lip structure configured on the lid.

FIG. 4 depicts structures and chemistry that may assist sensor function.

FIG. 5 shows that more than one chamber may be placed into an array device.

FIG. 6 is an example showing more than one cell sharing a chamber including an option for a specialty coating separating cells.

FIG. 7 shows one example of how the same type of cell may be treated differently within one chamber; an optional specialty coating is depicted separating cells.

FIG. 8 shows how patterned surface chemistries within the chamber may result in the ordered placement of distinct cell types within the chamber and/or how cells may be placed with desired chemistries and the subsequent cell function observed.

FIG. 9 is depiction of a single base surface with more than one cell, in which the lid is configured to create individual chambers for each cell upon closure and wherein a diffusional communication between chambers is enabled.

FIG. 10 is a drawing of a chamber holding multiple cells, or a cell culture or mono layer.

FIG. 11 depicts a sensor island within the lid structure in the context of multiple cells.

FIG. 12 shows the lid and base configured to further divide the device into chambers that share a geometrically-defined diffusional interface.

FIG. 13 shows details of configurations that provide structural support for larger chambers and the option for incorporation of diffusional restriction features between adjacent chambers.

FIG. 14 is a drawing of a base configured to allow for 3-D studies of cell cultures or tissue samples.

FIG. 15 is an embodiment of the chamber device in which intercellular columns support the lid structure.

FIG. 16 is a photograph of a 3×3 lid-on-top array with a Pt-OEP sensor, showing the position of the wells and lip of the chambers.

FIG. 17 is a photograph of a 3×3 lid-on-top array with a Pt-OEP sensor and wells containing cells.

FIG. 18 is a photograph of oxygen consumption (by cells) experiments in individual microwells showing fluorescence images of circumferentially deposited pH sensors within lids responding to microenvironmental changes (using CPC cells).

FIG. 19 shows various examples of multi-sensor mask designs configurations.

FIG. 20 shows examples of an optical analysis of multi-sensor lid configurations.

FIG. 21 depicts one example of a structural microgeometry for a multi-sensor configuration.

FIG. 22 illustrates examples of multi-sensor lid configurations after sensor deposition.

FIG. 23 shows confocal images of Pt-Porphyrin in a 3×3 micro-well array at different angles.

FIGS. 24A-F depict a microfabrication process for forming bottom substrates with wells according to one embodiment.

FIGS. 25A-L depict a microfabrication process for multi-sensor configuration lids according to one embodiment.

FIG. 26 illustrates chamber bases in an array coupled to a microfluidic device.

FIG. 27 illustrates a chamber lid configured with a tip that can be automatically loaded to a piston.

FIG. 28 illustrates one example of a chamber configuration according to one embodiment.

FIGS. 29A-F depict a closing mechanism of a chamber according to one embodiment.

FIG. 30 depicts the results of a seal test performed on a chamber of one embodiment of the present invention.

### DETAILED DESCRIPTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims. Unless defined otherwise, all technical and scientific terms used herein have the same meaning as those commonly understood to one of ordinary skill in the art to which this invention pertains.

As used herein and in the claims, the singular forms “a,” “an,” and “the” include the plural reference and equivalents known to those skilled in the art unless the context clearly indicates otherwise. Other than in the operating examples, or where otherwise indicated, all numbers expressing quantities of ingredients or reaction conditions used herein should be understood as modified in all instances by the term “about.”

All patents and other publications identified are incorporated herein by reference for the purpose of describing and disclosing, for example, the methodologies described in such publications that might be used in connection with the present invention, but are not to provide definitions of terms inconsistent with those presented herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on information available to the applicants and do not constitute any admission as to the correctness of the dates or contents of these documents.

The present invention provides a core technology that enables novel interrogation of living systems. The basic principle of operation is to place one or more cells in an chamber that can alternately be opened and closed. When open, the chamber can be perfused with any media or stimulus of choice. The present invention also enables the general confinement of cells of interest to predefined analysis locations, thereby enabling significant assay speed increases. When closed, the chamber is sealed and the depletion of, or accumulation of, molecules or characteristics of interest can be observed using sensors or sensor chemistries within the chamber. As such, the chamber enables the measurement of metabolic rates of production and consumption. Additionally, all methods of quantitative microscopy known in the art are simultaneously available within the scope of the present invention. For example, when the chamber materials are impermeable to oxygen and other gases, gas consumption and production rates can be measured. In other embodiments, the chamber lid material may be permeable to oxygen and impermeable to other molecules of interest (e.g., extracellular proteins), and therefore can be used to measure the buildup of these molecules with appropriate sensor selections. For example, sensors moieties may be deposited within a binding matrix and fixed to a location within the chamber, coated in a monolayer within the chamber, in free solution in their molecular form, and/or affixed to beads, to illustrate a few examples relevant to the embodiments of the present invention.

The invention addresses a critical need for high throughput assessment of cell physiology in multiple contexts and formats, with high precision and few invasive artifacts. The invention enables high throughput analysis of cell physiological response extending from individual living cells, 2D and 3D cell structures (artificial tissues), and tissue biopsies in a highly controllable micro-environmental context. The invention disclosed herein also addresses configurations that enable the study of intracellular communication via signaling molecules.

The embodiments described herein provide for the spatial deterministic location of cells distinct from sensor access locations using wells or patterned materials on the base; optimized seal lip geometries that can be demonstrated to have advantages over other random designs; multiple advanced sensor geometry and chemistries that enable previously unattainable dynamic range in measurement of extracellular species and the simultaneous measurement of multiple extracellular species of microenvironmental/microculture significance; compatibility by design with full automation of fluidic stimulus with measurement scan protocols; and compatibility with a full custom microenvironment control, cell placement, cell analysis, and end-point analysis pipeline.

The invention enables the performance of the above techniques using novel device geometries, integration of manufacturing process technologies, use of patterned material functionalizations and coatings, utilization of sensor deposition techniques, methods of device cassette manufacturing for automation, means of cell placement, and novel methods of measurement scanning The novelty of the present invention is significantly amplified by the plurality of permutations offered within the embodiments, as well as the unique enablement of dynamic range in the detection of molecules of interest.

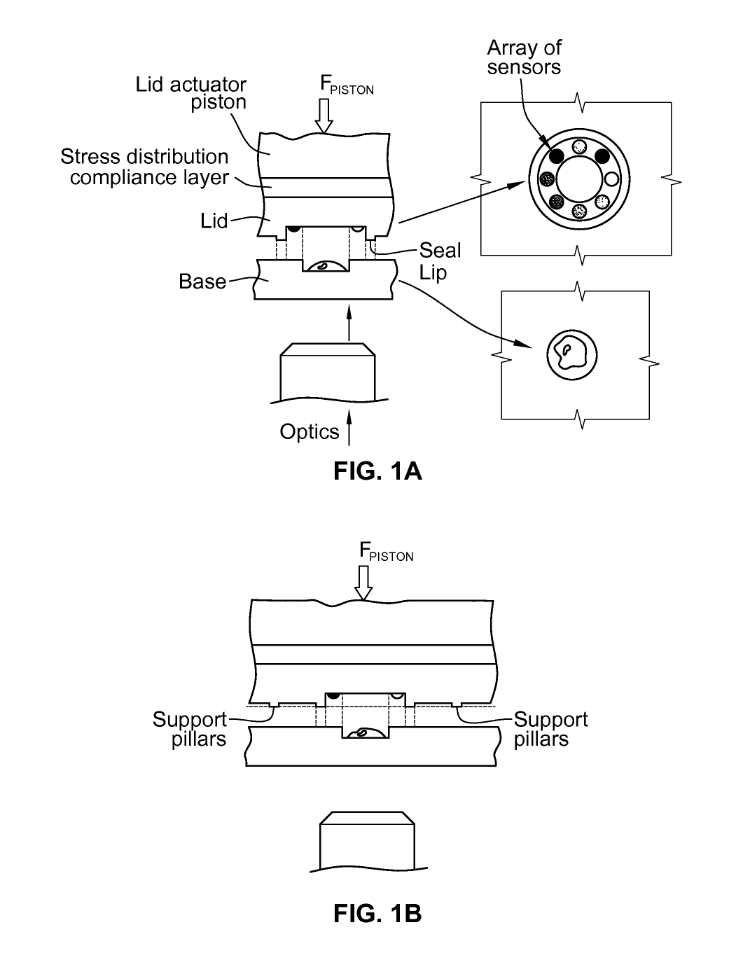

The present invention provides for a chamber for the placement and analysis of one or more living cells, or a living tissue, that can be alternately opened or closed to manipulate the microenviroment. The chamber confines the spatial location of the cell(s) for the purpose of observation of the intracellular and extracellular biological processes (e.g., genome, transcriptome, proteome, and physiome). The chamber may be comprised of a depression in a planar transparent material, and will typically have a characteristic breadth and depth dimension. The chamber is typically oriented to open upward, and may thus be considered as having a “base” of the device (FIG. 1A). It may be configured to contain one cell, multiple cells, or a tissue.

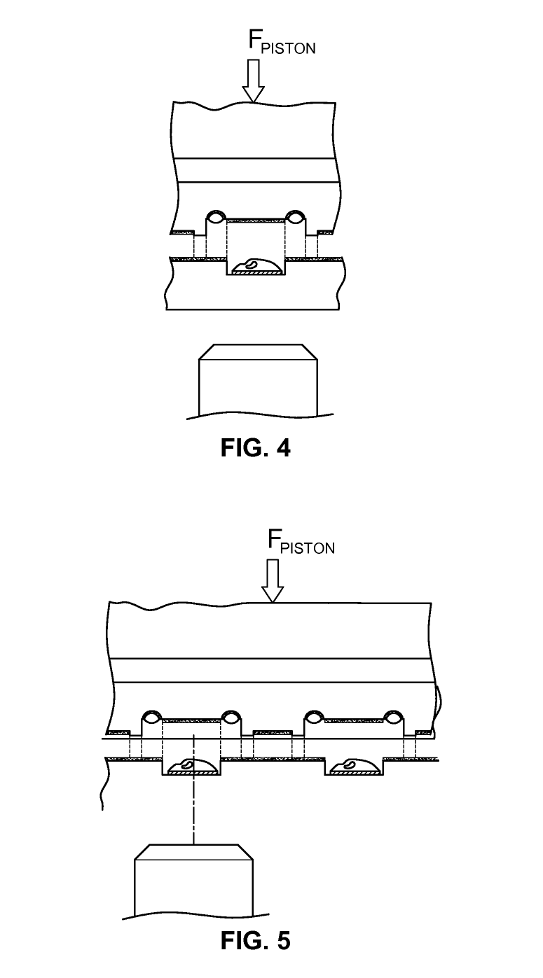

A second portion of the chamber, formed in a planar material, may be considered the “lid” for the “base.” The “lid” is typically of greater breadth than the base, and has width and height that encompasses the perimeter of the lid chamber. This perimeter, called a “seal lip”, has the function of providing a robust seal by concentrating seal pressure where it is needed when the lid is pressed against the base. The differential breadth of the lid in relation to the base is designed to provide spatial segregation between the sensors for measurement of extracellular species and the optical region occupied by the living cell(s) (FIG. 1A). In other words, the lid includes one or more sensors (that may be arranged in array) that are held above and spatially apart from the base structure which contains the living cell(s). The sensors may also be positioned outside the range of the optical view. The spatial segregation of the extracellular sensors from each other, and from the cell(s) of the chamber, enables full dynamic range control of sensor excitation light sources through the base, and emission collection sensors (FIG. 5). The lid may also include structures that serve as support pillars to provide stability to the lid as pressure is applied from above (e.g., by a piston) to seal the chamber (FIG. 1B). Further regarding the fabrication of the lid, geometry may be employed to enhance placement and fabrication of sensor arrays. The area of the lid fabricated to hold the sensor(s) may be treated with surface chemistry to enhance the sensor bond to the lid surface (FIG. 4).

The seal lip may be formed with feature height and width dimensions designed to maximize seal effectiveness with minimal force applied, optimized for the particular sensor well. The lid may also be coated with a substance that is inhibitory to cell and protein adhesion to prevent fouling (FIG. 3). Similarly, the region internal to the extent of the breadth of the base may be coated with an inhibitory compound to prevent protein adhesion.

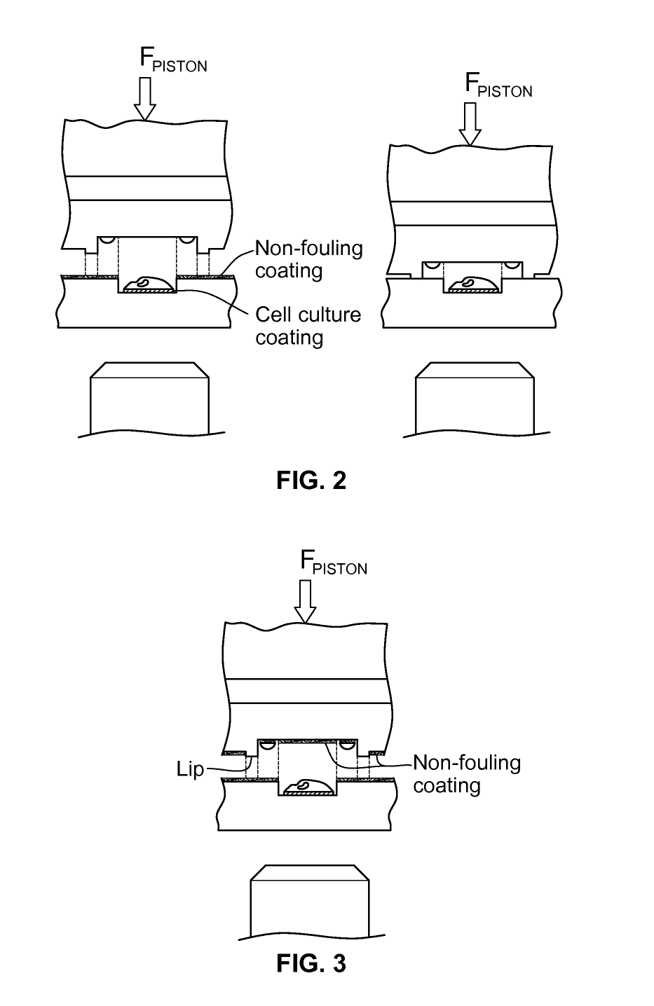

The bottom of the base of the chamber holds the living cell(s), and may be manufactured for optical sensing (FIG. 1). The cell chamber may be coated with a substance favorable to cell/tissue microculture for the purpose of encouraging a healthy microenvironment for cell study. It may also be coated outside the cell-holding portion, and the region internal to the extent of the breadth of the base may be coated with an inhibitory compound to prevent protein adhesion or with an inert substance that is inhibitory to cell adhesion to prevent fouling (FIG. 2). It is contemplated that the sensor(s) may additionally or alternatively be positioned in the base.

Additionally, the chamber may be configured such that an array of sensors that are sensitive to different molecules and compounds of interest are patterned in the region outside the breadth of the base chamber, but inside the breadth of the lid chamber, for the purpose of independent addressable sensor scanning with no excitation of the biological cells under examination. The sensors in the sensor array of the present embodiment may be calibrated using multivariate calibration techniques. These techniques, or other similar methods may be used in order to take advantage of multiple sensors with selected primary- and cross- sensitivities to moieties of interest.

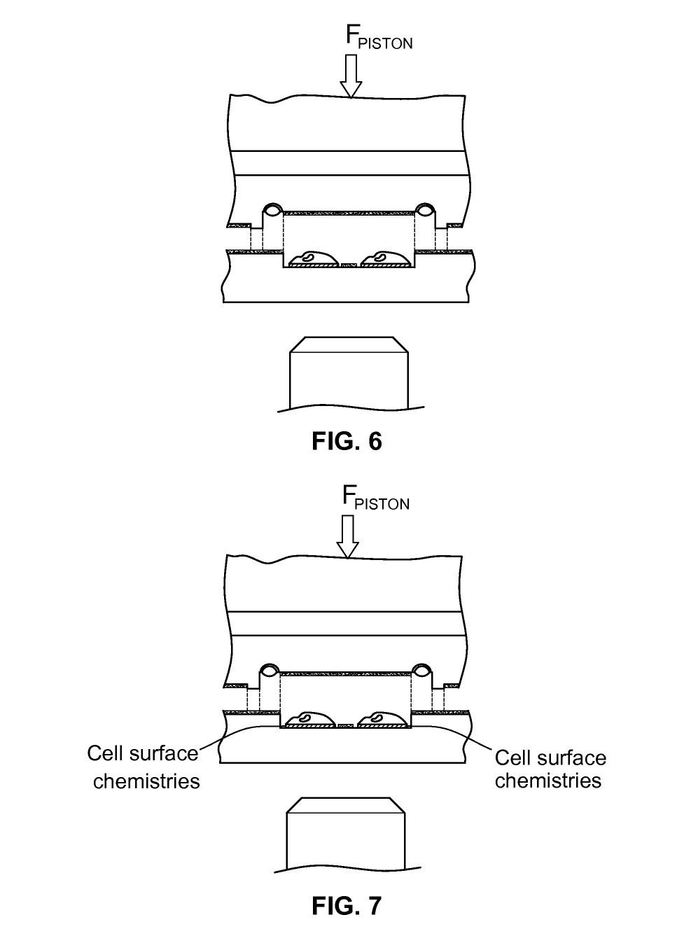

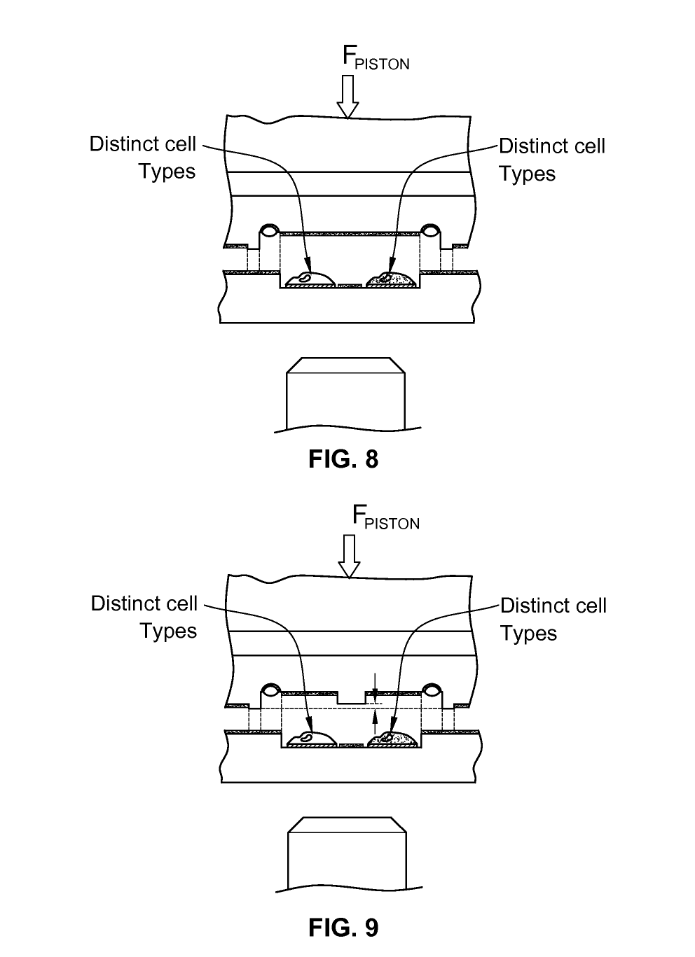

The system base and lid system described above may be configured whereby the base chamber size is increased to allow for more than one cell, and the lid chamber is increased in proportion to accommodate this increase in size. As shown in FIG. 6, two cells are affixed to array locations whereby cell culture enhancing/adhesion substance has been patterned in an array format. The cells are shown separated by a cell adhesion inhibiting patterned substance. Although two cells are shown in this image, and although patterned substances are shown, it is readily understood in the context of the invention that any permutation of these may be applied in array formats as required. One such permutation is illustrated in FIG. 7, where a different cell adhesion moiety is illustrated. FIG. 8 illustrates yet another permutation where patterned surface chemistries are used to create a pattern of distinct cell types. This embodiment is useful for the examination of cell to cell interactions, and cell to substrate interactions, both critical interaction studies in a plurality of life sciences disciplines. Thus, chamber surfaces patterned with distinct chemicals may be used to create a pattern of distinct cell reactions, and/or distinct cell types, within a single test chamber. In this fashion, the chamber is configured such that a structured pattern of substances may be placed in the bottom of the chamber for the purpose of building 2D cellular assemblies and/or test cell-to-substance deposited interactions (FIGS. 7 and 8).

The system embodied in FIG. 8 may also be made so that the chamber is divided into individual compartments that are connected to each other by a restriction that limits the rate of diffusion of molecules of interest between the chambers when the base and lid are closed (FIG. 9). As shown in FIG. 9, the chamber includes a diffusional restriction between the left and right chamber. This restriction is controlled by geometry and implementation of structure, and may allow for the diffusion of particular chemical species, but not the cells themselves, such that intercellular communication may be studied. The figure depicts the physical structure as a gap between the lid and base, but vertical slits or other selectable geometries could be employed.

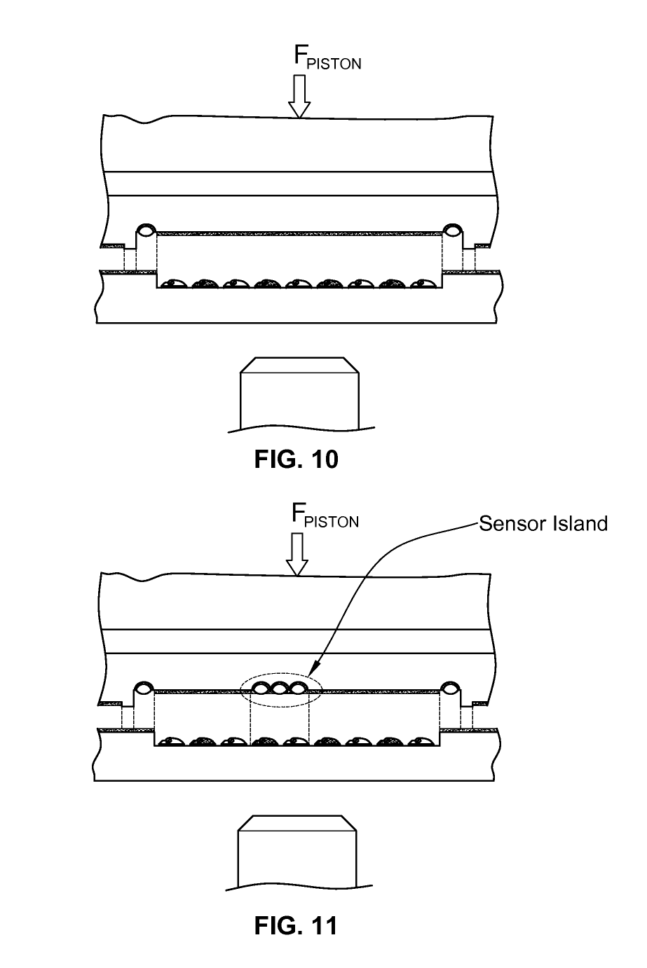

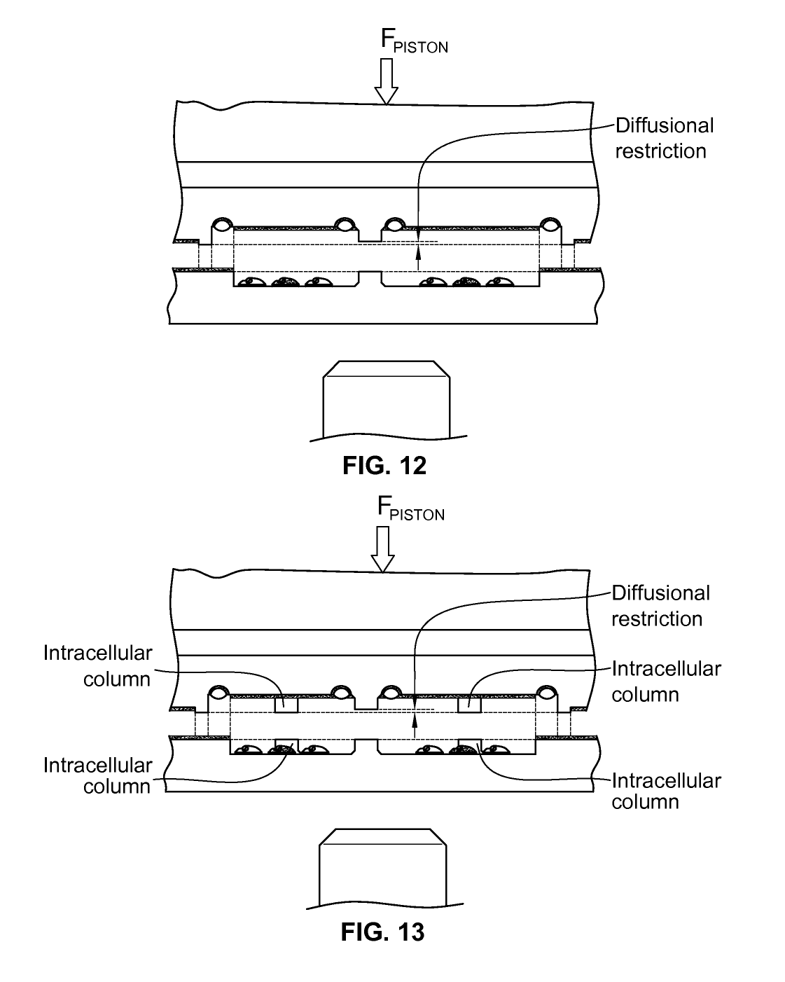

The fundamental base/lid/sensor design may also be used to observe cell populations in 2D formats of a suitable size (FIG. 10). Additionally, the system may be used to create a 2D pattern of cells by patterning the base chamber surface in an arrayed format with cells then placed in this arrayed format. This larger device of FIG. 10 may include auxiliary sensor array patterns placed at intermediate locations within the lid, and the cells may be excluded from the base in these regions by any of a number of methods (FIG. 11). Also, the larger format device may include a diffusion restriction element, incorporated for physiology studies whereby cells within one chamber communicate with cells in the other chamber via intercellular communication molecules (FIG. 12). Regarding the structure of the device, the larger format device may have “support pillar columns” which are incorporated into the microfabricated device design in order to insure uniform pressure and stress distribution and proper device sealing (see FIG. 13).

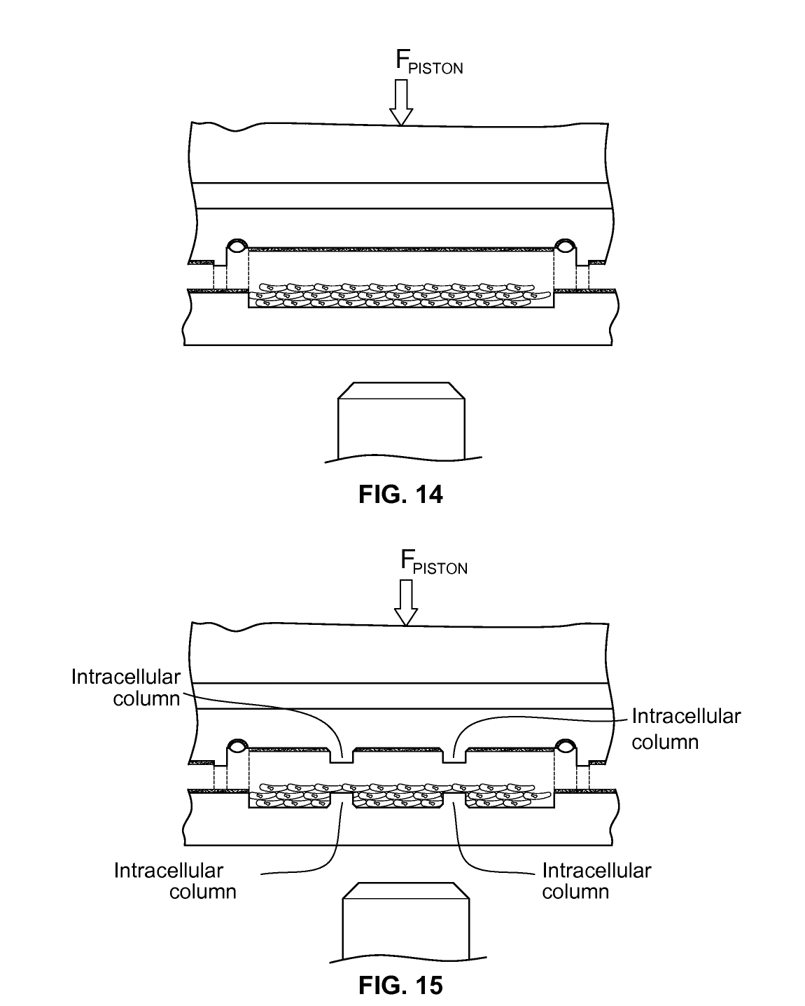

The larger format device may also be configured for and used in the study of 3D cell structures, either randomly assembled in layers, or assembled using deterministically placed cells with deterministically placed cell matrix cofactors, as one of many examples (FIG. 14). This allows for the design of ordered stromal structures such as artificial tissues with interjected scaffolds incorporated by extension, either patterned/structured or randomly placed. Additionally, this embodiment may have column pillars/supports added to enhance device sealing function and structural stability of the device (FIG. 15).

The chamber device base is generally constructed from a highly oxygen and carbon dioxide impermeable material that is simultaneously impermeable to biological molecules of interest. The lid may also be constructed with associated functional features and sensors may be reused many times prior to completion of a useful service life. Alternatively, the lid is single-use and is disposable, and may be specifically designed and manufactured for automated loading and automated disposal. The chamber material may also be fabricated from any transparent gas impermeable material, such as glass and quartz, for the measurement of gas consumption and production. Gas permeable materials may be used when the intent is to perfuse the cells while measuring the depletion of larger molecules of interest, or the production of larger biomacromolecules (e.g., proteins).

The chamber of the present invention is compatible with systems including partially or fully automated fluidic stimulus with measurement scan protocols. See, e.g., Dragavon et al., J. R. Soc. Interface, Jun. 27 (2008). The present embodiments are ideally suited for simultaneous measurement of intracellular and extracellular physiological responses of living cells to external stimuli using optical or electronic sensor transduction means.

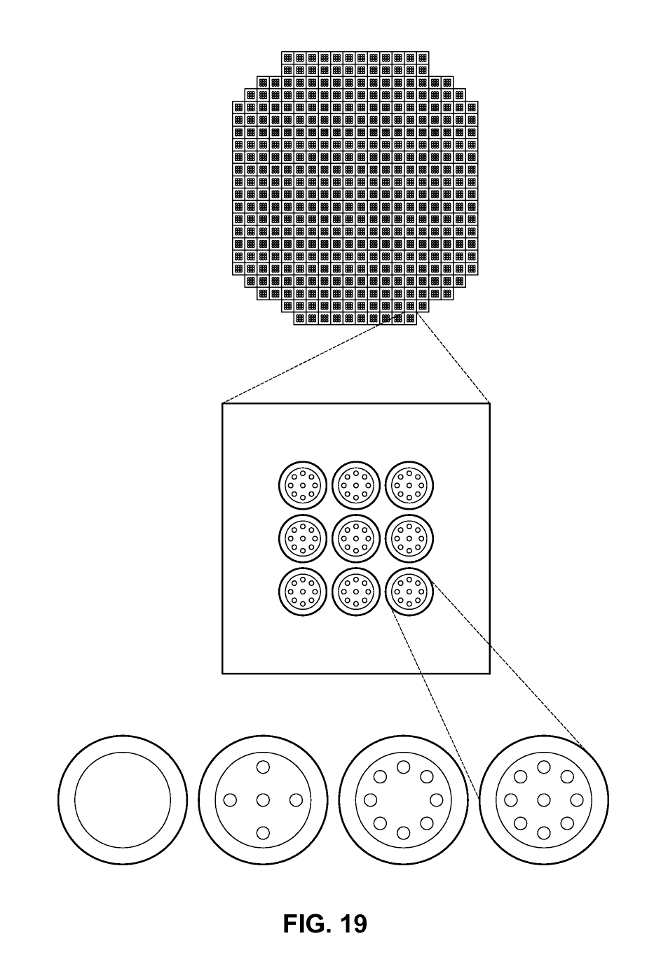

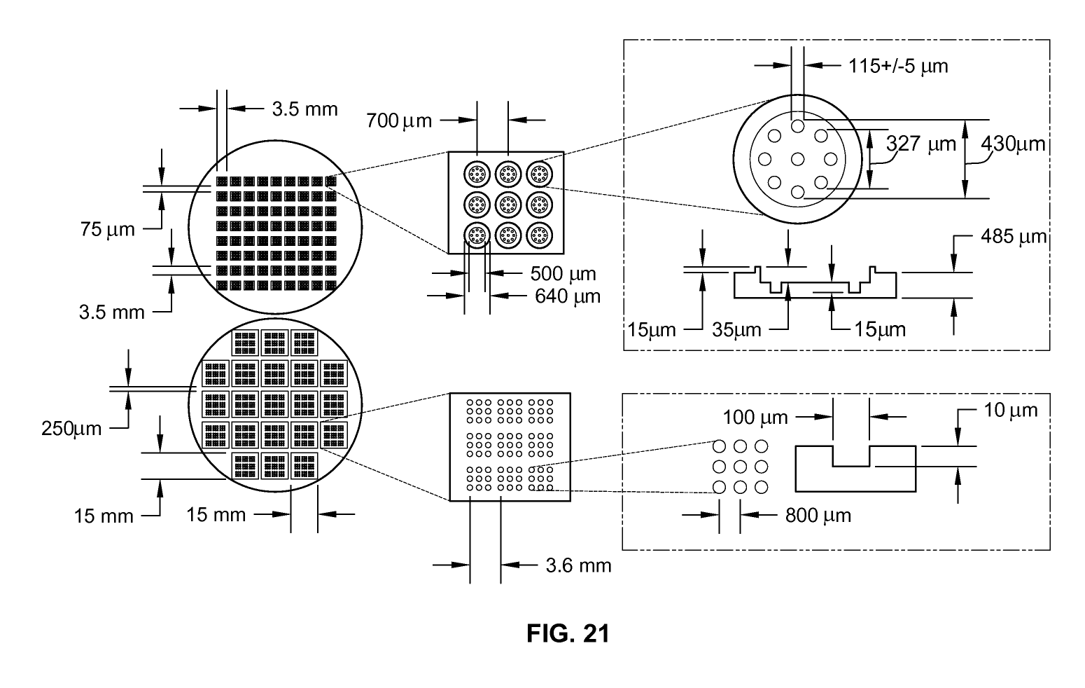

As discussed above, lids of various designs may accommodate a range of sensors. The various lid designs may vary based on the number of sensor deposition pockets. Sensor deposition pockets are small pockets that are fabricated to receive a sensor that enables spatial confinement of the sensor material, thereby enabling one method for manufacturing sensors in lids. The surface tension works to self align the sensor in the receiving pocket geometry so long as the sensor is aligned well enough to be received in the pocket. FIG. 19 includes illustrative pictures of various mask designs having different numbers of sensor deposition pockets. The masks of FIG. 19 were prepared to accommodate a set of demonstration embodiment designs. As also shown in a close-up view of FIG. 21, each square block (top left) of FIG. 19 is a substrate of a 3×3 array of lids. Each substrate has a different configuration of lids. The bottom four pictures in FIG. 19 illustrate various examples of designs that may be realized. However, depending upon the number of sensors, any number of designs can be realized.

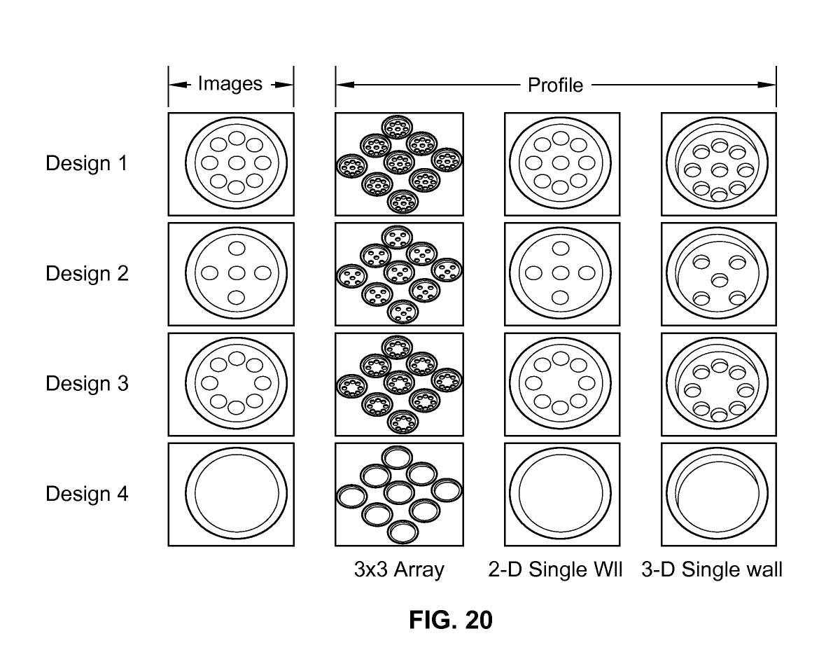

FIG. 20 shows optical images of the different designs of FIG. 19. The first column of images was obtained by a microscope. The remaining columns of images were obtained by a non-contact optical profilometer (Hyphenated Systems' H-S 200 Advanced Confocal Profiling System, Burlingame, Calif.). As shown, design 1 has nine sensor deposition pockets that can accommodate nine sensors, design 2 has five sensor deposition pockets to accommodate five sensors, design 3 has eight sensor deposition pockets to accommodate eight sensors, and design 4 has only one sensor deposition pocket to accommodate a single sensor. The various sensors may be the same types of sensors, different types of sensors, or a combination thereof. In general, where only a single sensor (e.g., design 4) is to be accommodated, the geometrical dimensions of the lid are relatively small (as compared, e.g., to designs 1 thru 3).

FIG. 21 shows the geometrical dimensions of multi-sensor array lids (e.g., design 1 of FIG. 20) and microwells according to one embodiment. In the chamber configuration of the disclosed embodiments, the sensor and live cell are not in contact, as the cell resides in the bottom well and the sensor resides in the top well.

As described above, in order to accommodate a different number of sensors, each lid is fabricated with a different number of pockets that can accommodate a sensor material. In the example of FIG. 21, each lid has nine pockets and can accommodate nine different sensors. The diameter of each pocket is about 115±5 μm with an about 15 μm depth. All of the pockets are designed to be within a circle having a diameter of about 430 μm.

The geometrical description of micro-wells. The “lid on top” configuration, is defined to be where the cells reside in the bottom well, and the lid with the sensor(s) is placed on top of the microwell. In one example, each well has a diameter of about 100 μm with a depth of about 10 μm. Each microwell is separated by a distance of about 800 μm.

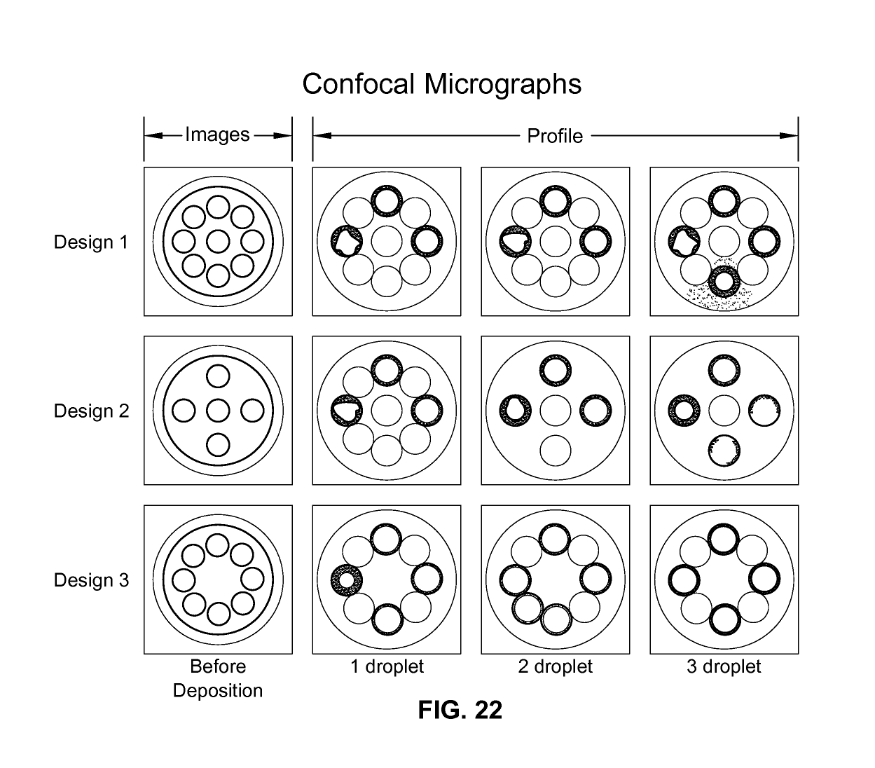

FIG. 22 illustrates various designs of the multi-sensor array lid before and after sensor deposition. The first column includes microscopic images of various lid designs before sensor deposition. As evident in FIG. 22 and as described above, the number of pockets is different in different designs to accommodate a range of sensors. The lids are deposited with various sensors using, for example, a piezodispensing device. The lids, having various droplets of a compound (e.g., a viscous polymer) dispensed thereon (as shown in FIG. 22) may then be excited at their absorption bands using a confocal microscope. The confocal micrographs (see columns 2-4 of FIG. 22) show various sensors emitting at different wavelengths. The diagonal pockets are deposited, and the remaining pockets are left empty to demonstrate the effectiveness of the multi-sensor array lid configuration. Each column of confocal micrographs has been deposited with a different amount of sensor material, as evidenced by their respective emitting intensities.

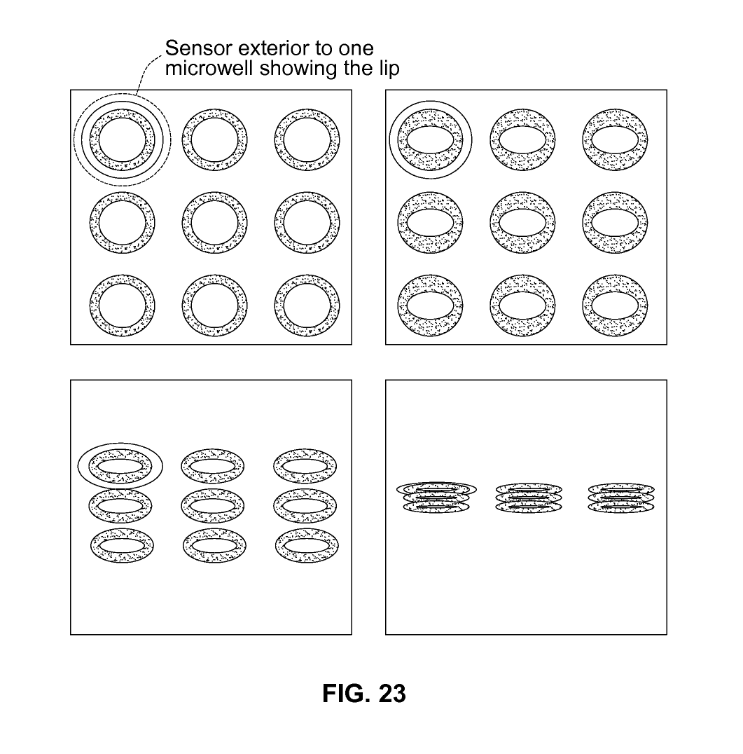

FIG. 23 shows confocal images of a substrate with a 3×3 lid and rotated at different angles in order to demonstrate sensor emission as well as the structure. As shown in FIG. 23, the lip of the lid is clearly visible (circled in the first image) and the sensor deposited in the lid is circumferentially cured due to surface tension.

FIG. 28 shows a schematic view of a chamber configuration according to one embodiment. As shown in FIG. 28, in this configuration, a sensor resides in a top well while a live cell resides in the bottom well. Depending on the fabrication process, the lid/wells are either isotropic or anisitropic. FIG. 28 illustrates a structure fabricated using wet etch process resulting in an isotropic (semicircular) chamber. The arched segment in the lid represents the sensor material deposition. One advantage of this configuration is that the cell is not in the vicinity of the sensor material and is immune to the effects of sensor characteristics.

Once the lid is closed on top of the well, a hermetic microenvironment is formed. This sealed chamber with sensor may be excited using a broadband source. The intensity of the sensor is monitored over time. As the emitting intensity is a function of the concentration of the analyte, it gives an accurate estimation of analyte concentration within the microenvironment. Together, the bottom well (with cell) and top well (with sensor) result in a hermetically sealed microenvironment. These wells with sensors in them will be employed as lids to monitor the moieties of interest.

The lid is attached to the piston using a compliant layer. This layer ensures the even distribution of force throughout the surface of the lid. The compliant layer can be any material such as PDMS, with properties of adhesion in order to hold the lid on one side and to stick onto the piston on the other side. The piston is fixed to an xyz manipulator and a rotator. An inverted microscope with data acquisition (in-house customised Nikon TE) capability is employed for analysis. The piston is generally lowered until the lip of the lid touches the bottom substrate. The micrograph shows the image after the lids are closed on top of the wells. The seal lip and the cells in the wells are identified. As mentioned earlier, the dimensions of the lid and wells may vary. However, achieving a hermetically sealed microenvironment with the same diameters of lids and wells is also contemplated. The micrograph on the right-hand side of FIG. 28 is an actual image of cells within the wells and closed by a lid. The various arrows show the lid, lip and cells. Each chamber is loaded with a single cell. However, some of the chambers might have two cells, which is the resultant of incubation time after the loading. It is quite common for the cells to divide over incubation.

Depending on the number of analytes to be monitored, the chamber configuration of the illustrated embodiments can be broadly classified into two main categories: one having a single sensor and other having multiple sensors. In single-sensor technology, only one sensor (e.g., an O₂ sensor) will be deposited in the lid. This type of lid monitors only one analyte at any time. In multi-sensor configurations, simultaneous measurement of different analytes can be performed at any point of time. However, the dimensions of both the lids and wells in multi-sensor configurations may be greater than in single-sensor configurations.

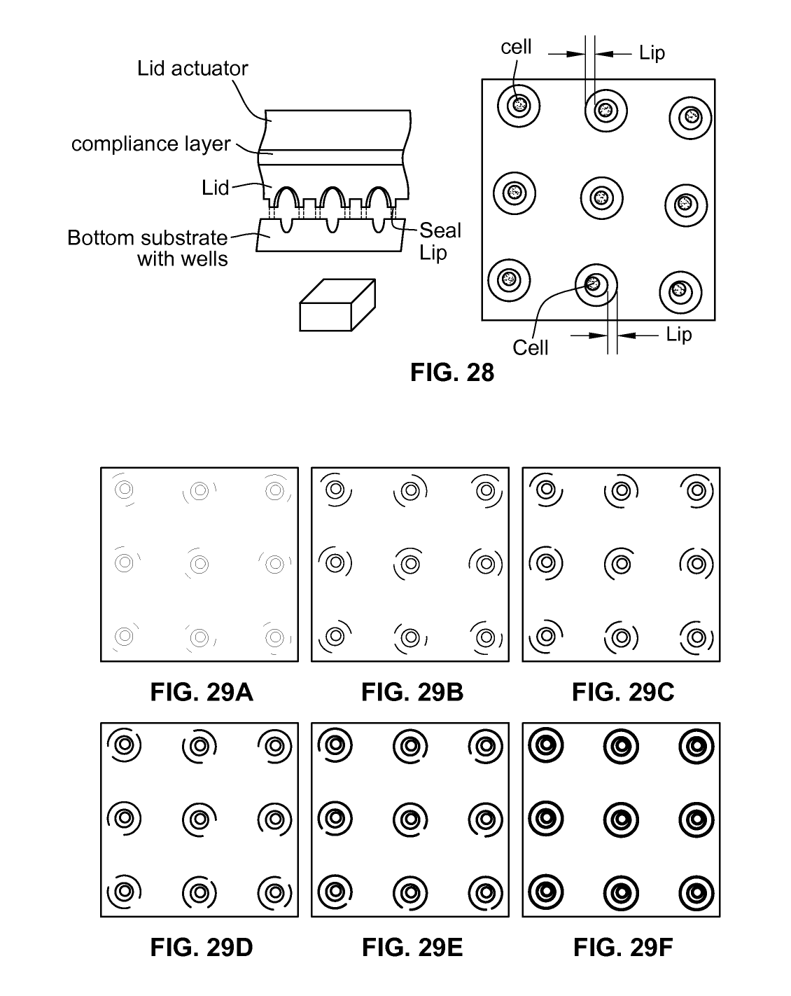

FIG. 29A-F show a sequence of steps involved in achieving the hermatic seal. Initially the microwells are imaged and the focal plane is fixed on the microwells (FIG. 29A). The piston with lid is lowered until it reaches the focal plane focused at the microwells (FIG. 29B). At this point, very often the lid is not alligned and needs angular adjustment. The lid is then rotated in order to accurately allign the lids with wells (FIGS. 29C, 29D). Once the wells and the lid are alligned, the piston is lowered completely (FIGS. 29E, 29F) using the xyz manipulator. A weight is placed on top of the piston to achieve a sealed microenvironment contained in a chamber with the lid on top of the microwell. Since the piston is a metal, scattered light can be noticed in the background. However, the background along with any scattered light from outside will be appropriately treated in data acquisition.

Once the lid is closed, a hermatically-sealed environment is created within the confines of the well on the bottom substrate and the lid of the top substrate. In order to prove the efficacy of the configuration, a seal test may be performed. To perform the seal test, a substrate with wells is placed in a Petri dish with about 3-5 ml of buffer/media/water. As explained above, the lid is aligned and placed on top of the wells. A nitrogen channel is placed in the aqueous solution. This channel is then employed to strip the dissolved oxygen in the media. Once the lid is placed, the seal lip generates a diffusion resistant barrier between the chamber and outside media. A LabView data acquisition program (custom built) may begin collecting the intensity data.

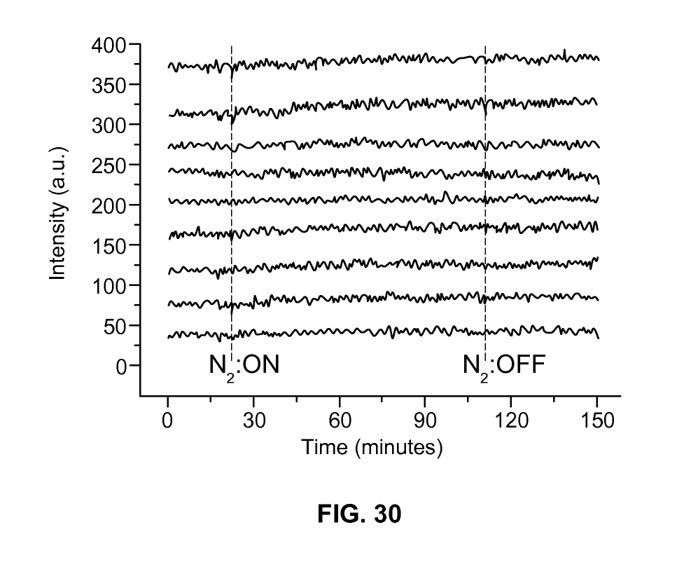

FIG. 30 shows the intensity curves from all nine wells. The curves were manually shifted for the purpose of illustration. This test is performed on multi-sensor lids (e.g., Design 2 of FIG. 20). Initially the intensity profiles are monitored for approximately 30-40 minutes. This step is performed to account for intensity profile fluctuation that may occur during the process of equilibration between the sensor and the media. Once the equilibrium is achieved (no change in their intensity), nitrogen is switched on. This event is denoted by “N₂ ON.” Dissolved oxygen present in the aqueous solution may be removed by nitrogen bubbling. The nitrogen bubbling may takes place within the Petri dish external the microenvironment generated by chamber configuration. However, in the case of minute leaks at the interface of seal lip of lid and bottom substrate, the dissolved oxygen present within the microenvironment starts leaking to an external Petri dish environment due to diffusion. The sensor within the microenvironment responds to the outward diffusion of dissolved oxygen from the microenvironment. This is reflected by an increase in the intensity of the sensor. The start of nitrogen bubbling is identified by “N₂ ON” in FIG. 30. It is evident from FIG. 30 that none of the curves respond to the nitrogen bubbling. This demonstrates that a hermetic seal is achieved by placing lids on top of wells.

### EXAMPLES

### Example 1

### Live Cell Chamber with Photoluminescence Array

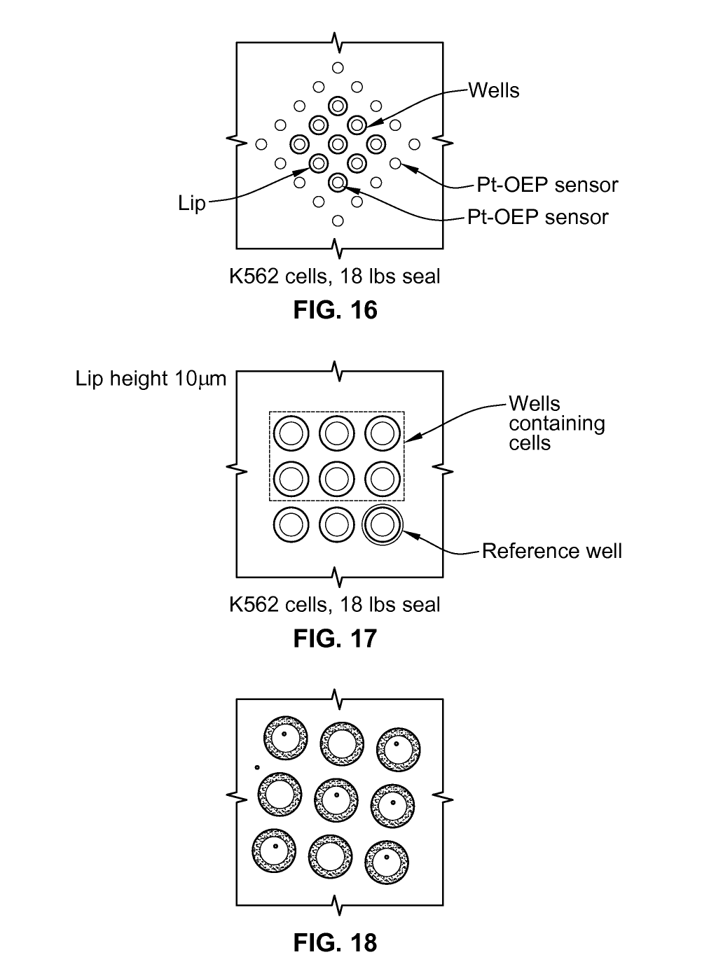

An embodiment of the present chamber was fabricated with a 3×3 lid-on-top array comprising platinum octaethyl porphine (Pt-OEP), a photoluminescent dye that serves as an oxygen-sensitive probe in biological samples. K562, human immortalised myelogenous leukaemia cells, were placed in the chamber wells, and the lid sealed with 18 lbs pressure. FIG. 16 is a micrograph showing a view of the chamber wells, lip, and Pt-OEP sensors through the optically transparent base. FIG. 17 shows the luminescence of the Pt-OEP sensors as photographed from below.

### Example 2

### Drawdown Experiment

FIG. 18 shows the fluorescence image of pH sensor lids in the presence of calcium preconditioned (CPC) cells.

### Example 3

### Microfabrication of Substrates with Wells

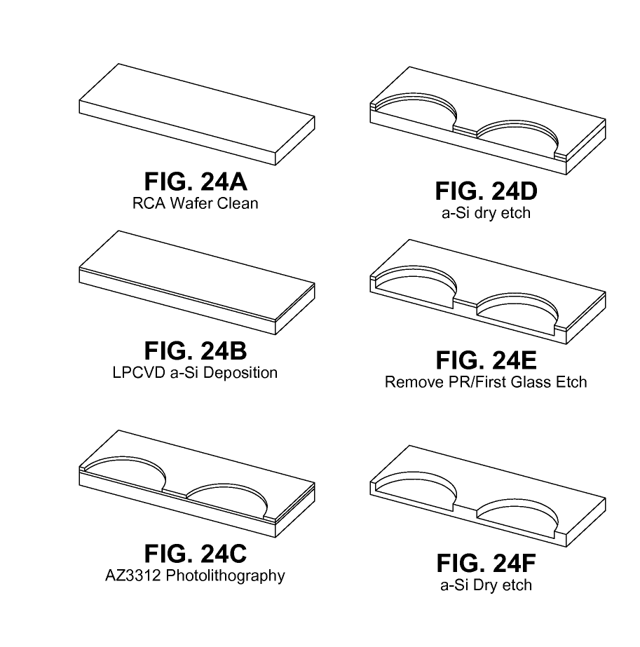

According to one example shown in FIGS. 24A-F, a process of microfabricating a well positioned on the base of the device began with RCA cleaning of the 4 inch double-side polished fused silica wafers (Mark-Optics, Santa Ana, Calif.) to free the substrates of organic and inorganic contamination (FIG. 24A). 2000 Å amorphous silicon was then deposited onto the substrates as the masking layer by using a Chemical Vapor Deposition (CVD) technique (300 mT, 560° C., 60 sccm SiH₄) (FIG. 24B). Amorphous silicon (a-Si) was selected in the illustrated example because it shows the least number of pin-holes and notching effect during the subsequent wet etching. 1 μm positive photoresist AZ 3312 was then spin-coated and patterned onto the substrate using standard photolithography technique (FIG. 24C), which defines the micro-well. The Reactive Ion Etch (RIE) dry etch was then performed (FIG. 24D) to transfer the pattern into the a-Si layer, and the photoresist was then removed using a microstripper, as shown in FIG. 24E. The wet etch was used to etch the micro-well down to 10 μm for the cell deposition. The remaining a-Si was removed by the fourth RIE dry etch to finish the device micro-fabrication. The finished wafer was then cut into designed geometry using a standard dicing saw.

### Example 4

### Microfabrication of Multi-Sensor Configuration Lids

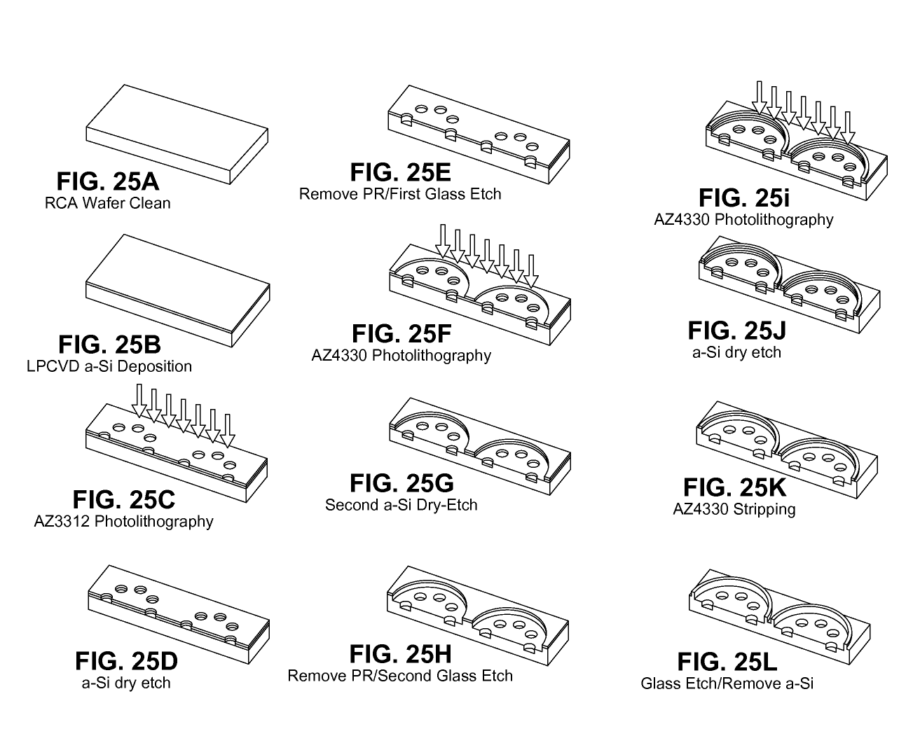

FIGS. 25A-L illustrate an example of the microfabrication flow process for multi-sensor lids. According to the illustrated example, the process began with RCA cleaning of the 4-inch double-sided polished fused silica wafers (Mark-Optics, Santa Ana, Calif.) to free the substrate surface of organic and inorganic contamination (FIG. 25A). 2000-5000 Å amorphous silicon was then deposited onto the substrates as the masking layer by using CVD technique (FIG. 25B). Amorphous silicon was selected because it shows the best interface adhesion and lower film stress during the subsequent wet etching and thus, fewer pinhole defects. Other silicon-based thin film (e.g., silicon nitride, silicon carbide, and poly-silicon) with similar properties can also serve as the masking layer. 1 μm positive photoresist AZ 3312 was then spin-coated and patterned onto the substrate using a standard contact photolithography technique (FIG. 25C), which defines the micro-pocket inside the micro-well. RIE was then performed (FIG. 25D) to transfer the pattern into the a-Si layer, and the photoresist was then removed by a microstripper, as shown in FIG. 25E. The first wet etch was used to etch the micropocket down to 15 μm for the subsequent sensor deposition. A second photolithography step using a thicker photoresist was performed to define the microwell layer (FIG. 25F). 3 μm AZ 4330 was used (instead of 1 μm AZ 3312) to acquire a uniform coating and better step coverage across the whole wafer. Other photoresist with over 1 μm thickness may also be used in this step, such as, for example, AZ 4620, or Shipley 1825. A second RIE a-Si dry etch was then performed to transfer the micro-well pattern into the remaining a-Si layer (FIG. 25G). AZ 4330 was then removed by a microstripper, and a second HF wet etch was used to etch a 35 μm deep micro-well (FIG. 25H). Since the wet etch is isotropic and the micropocket was exposed to the second wet etch, the geometry of the micropocket will remain 15 μm deep in relation to the microwell bottom during this etch process. That is, the sensor deposition pocket etches at the same rate at the bottom of the microwell. The third AZ 4330 photolithography was then performed to define the final lip layer (FIG. 25I). A third a-Si dry etch (FIG. 25J) HF glass etch was performed to transfer the lip pattern to the substrate. The remaining a-Si was removed by the fourth RIE double side dry etch (FIG. 25L) to finish the device microfabrication. Finished wafers were then cut into designed chip geometry using a standard dicing saw.

### Example 5

### Interface to Automated Systems

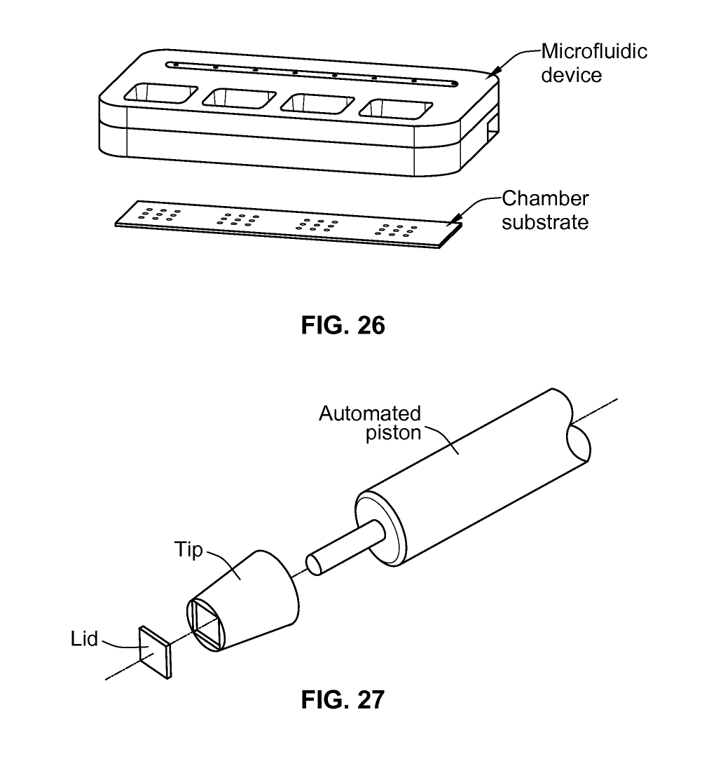

FIG. 26 illustrates a microfluidic device that has been designed to couple with a substrate upon which the chamber bases have been microfabricated in array format. The cassette has features that enable robotic manipulation.

FIG. 27 illustrates a method of mounting the chamber lids (having sensor arrays in microwells) on a tip that has features that enable robotic manipulation (tip load and unload).

Each of these embodiments and obvious variations thereof is contemplated as falling within the spirit and scope of the claimed invention, which is set forth in the following claims.

1. A chamber device for analyzing living cell(s) comprising:
a base and a lid that when reversibly pressed closed create a chamber;
the base configured with an optically transparent well to contain at least one cell;
the lid having a breadth greater than the base, and configured to contain at least one sensor;
wherein the lid is further configured with a lip that when pressed between the lid and the base creates an impermeable seal; and
wherein the base and the lid are configured so that, when closed and in use, the sensor remains spatially apart from the at least one cell.

2. The chamber of claim 1, wherein the base is treated with at least one chemical that effects cell function.

3. The chamber of claim 1, wherein the base is treated with at least two chemicals that are applied in a predetermined fashion to form a pattern.

4. The chamber of claim 1, wherein the at least one sensor is located in at least one of a corresponding at least one pocket that is fabricated to receive the at least one sensor, the lid, and the base.

5-7. (canceled)

8. The chamber of claim 1, wherein the at least one sensor is located outside the breadth of the well.

9. (canceled)

10. The chamber of claim 1, wherein portions of the lid and the base external to the well are treated with a substance that inhibits protein adhesion.

11. The chamber of claim 1, further comprising support pillars configured to assist in ensuring uniform pressure and stress distribution and proper sealing of the base and the lid.

12. The chamber of claim 1, further comprising a plurality of chambers formed by one or more diffusion restriction elements, the diffusion restriction elements allowing diffusion of chemicals, but not cells, between the plurality of chambers.

13. (canceled)

14. The chamber of claim 1, wherein at least one of the lid and the base is made from a material that is highly oxygen and carbon dioxide permeable, and impermeable or selectively permeable to other biological molecules.

15.-17. (canceled)

18. The chamber of claim 1, further comprising a microfluidic device to which the base is mounted.

19. The chamber of claim 1, wherein the base is configured to contain a plurality of cells.

20-22. (canceled)

23. A chamber for analyzing at least one cell, the chamber including:
a base having an optically transparent well configured to include the at least one cell; and
a lid including a plurality of sensors, the plurality of sensors being spatially segregated from one another and from the at least one cell;
wherein the base and the lid are coupled to form a seal when the chamber is in a closed position.

24. (canceled)

25. The chamber of claim 23, further comprising a mechanism for dividing the chamber into individual compartments.

26. The chamber of claim 25, wherein each of the individual compartments is connected to at least one other individual compartment by a restriction that limits the rate of diffusion of molecules of interest when the chamber is in the closed position.

27. The chamber of claim 23, further comprising support pillars configured to assist in ensuring uniform pressure and stress distribution and proper sealing of the base and the lid.

28. The chamber of claim 23, further comprising a plurality of chambers formed by one or more diffusion restriction elements, the diffusion restriction elements allowing diffusion of chemicals, but not cells, between the plurality of chambers.

29. The chamber of claim 23, wherein each of the plurality of sensors is spatially separate from one another and separate from the cells in the optical viewing plane.

30. The chamber of claim 23, wherein at least one of the lid material and the base material is selectively permeable to a moiety of interest.

31.-32. (canceled)

33. The chamber of claim 23, wherein the lid has a greater breadth than the base, and has a width and height that encompasses the perimeter of the lid, thereby forming a lip on the lid.

34. (canceled)

35. The chamber of claim 33, wherein the plurality of sensors is patterned in a region outside the breadth of the base and inside the breadth of the lid.

36. The chamber of claim 23, wherein the plurality of sensors are located in a corresponding plurality of pockets that are fabricated to receive each sensor.

37. (canceled)

38. The chamber of claim 23, further comprising a patterned surface chemistry applied to the base to create a pattern of distinct cell types.

39. The chamber of claim 23, wherein the base is formed of a material that is permeable to oxygen and carbon dioxide.

40. (canceled)

41. The chamber of claim 23, wherein the base further includes a structured pattern of cells creating a two-dimensional cellular assembly.

42-45. (canceled)

46. The chamber of claim 23, further comprising a microfluidic device to which the base is mounted.
